(19) United States
(12) Patent Application Publication
(10) Pub. No.: US 2006/0094009 A1
(43) Pub. Date: May 4, 2006

(54) Method for the characterisation of nucleic acid molecules

(76) Inventors: Patrick Martin Vaughan, Glenageary (IE); Thomas Valentine McCarthy, Montenotte (IE)

Correspondence Address: BIRCH STEWART KOLASCH & BIRCH, PO BOX 747, FALLS CHURCH, VA, 22040-0747, US

(21) Appl. No.: 10/519,153

(22) PCT Filed: Jun. 25, 2003
(86) PCT No.: PCT/IE03/00098

§ 371 Date: Sep. 21, 2005

(30) Foreign Application Priority Data
Jun. 28, 2002 (IE) 2002/0544

(51) Int. Cl. C12Q 1/68; C12P 19/34

(52) U.S. Cl. 435/6; 435/91.2

(57) ABSTRACT

A method for characterising nucleic acid molecules comprises the steps of i) introducing a modified base which is a substrate for a DNA endonuclease, for example Endonuclease V from E. coli, into a target DNA molecule; and ii) reacting the nucleic acid containing the modified base with said DNA endonuclease such that the nucleic acid is cleaved to generate an upstream fragment condition said modified base and bearing a 3′ hydroxyl group. The upstream fragment generated in step ii) can be used as a primer for a subsequent extension reaction. The modified base as such targets cleavage of the nucleic acid strand because it directs the endonuclease to cut the nucleic acid strand on which it resides at a site predominantly one nucleotide removed therefrom, so that the modified base remains in the nucleci acid as th upstream fragment. The method can be used for detecting polymorphisms and mutations and for nucleic acid indentification and profiling. The method enables one to detect polymorphisms in any sequence context and such detection is not dependent on surrounding nucleotide sequence.

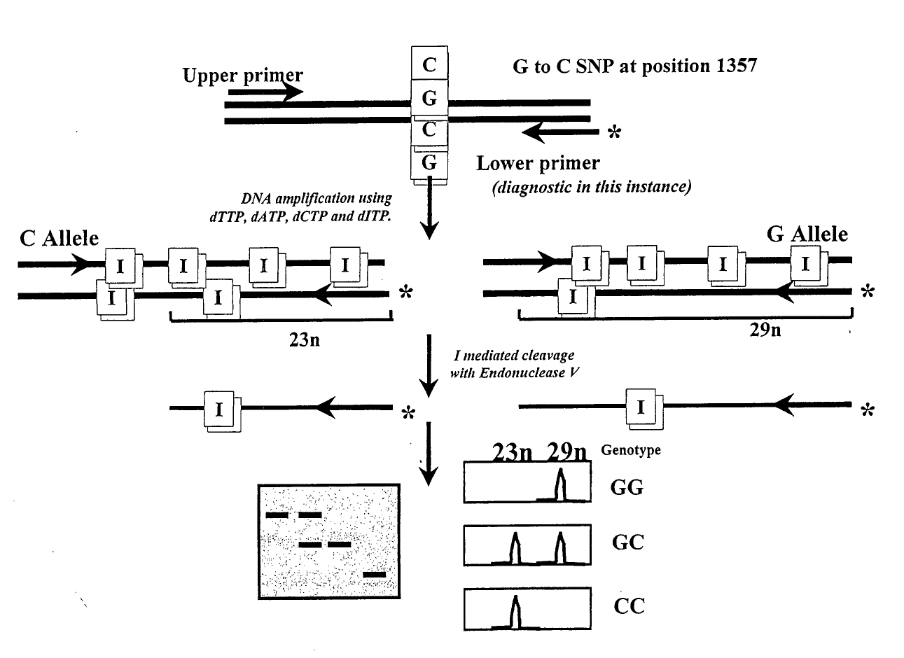

### TECHNICAL FIELD

This invention relates to a method for the rapid and sensitive characterisation of nucleic acids, especially DNA, including the identification and detection of genetic variations in a target nucleic acid.

### BACKGROUND ART

Detection of specific sequences at given loci in nucleic acids is central to many diagnostics procedures, notably in the diagnosis of inherited disorders and of infectious diseases. On-going requirements for such diagnostics procedures include optimising high sample throughput and sensitivity and specificity.

WO 97/03210 describes a method for rapidly detecting the presence or absence of a particular nucleic acid sequence at a candidate locus in a target nucleic acid sample which comprises the steps of: i) introducing a modified base which is a substrate for a DNA glycosylase into the candidate locus at one or more preselected positions; ii) excising the modified base by means of said DNA glycosylase so as to generate an abasic site; iii) cleaving phosphate linkages at abasic sites generated in step ii); and iv) analysing the cleavage products of step iii) so as to identify in said target nucleic acid sequence the presence or absence of the particular nucleic acid sequence at the candidate locus.

WO 99/54501 describes a method for characterising nucleic acid molecules which involves generating extendible upstream DNA fragments which result from the cleavage of nucleic acid at an abasic site involving the use of DNA glycosylase. The extendible upstream fragment is incubated in the presence of an enzyme, for example a polymerase or a ligase, allowing for extension thereof and template nucleic acid followed by analysis of the resultant fragment(s). This method can be used to scan or check a fragment of DNA for the presence or absence of a mutation.

Any method which reduces the time taken, while at the same time improving sensitivity, for characterising specific sequences would have significant advantages in the detection of mutations and also for genotyping known polymorphisms and mutations.

### DISCLOSURE OF INVENTION

The invention provides a method for characterising nucleic acid molecules, which comprises the steps of:
i) introducing a modified base which is a substrate for a DNA endonuclease into a target DNA molecule; and
ii) reacting the nucleic acid containing the modified base with said DNA endonuclease such that the nucleic acid is cleaved to generate an upstream fragment containing said modified base and bearing a 3′ hydroxyl group.

The nucleic acid can be characterised at this stage by analysing the cleavage products in a manner known per se.

The invention has advantages over the prior art methods described above, including the fact that a secondary enzymatic or chemical cleavage step is not required, thereby allowing higher throughput and more cost-effective DNA characterisation. Thus, the method according to the invention involves endonuclease mediated cleavage rather than glycosylase mediated cleavage as in the case of the prior art methods described above.

In the method according to the invention the modified base as such targets cleavage of the nucleic acid strand because it directs the endonuclease to cut the nucleic acid strand on which it resides at a site predominantly one nucleotide removed downstream therefrom, so that the modified base remains in the nucleic acid as the upstream fragment. Therefore, if an upstream fragment is generated that is 25 nucleotides long, then the modified base is at position 24 (counting 5′ to 3′) of said fragment.

Furthermore, the method according to the invention enables one to detect polymorphisms in any sequence context and such detection is not dependent on surrounding nucleotide sequence.

Preferably, the modified base is introduced by enzymatic amplification of the nucleic acid.

In this embodiment, the target nucleic acid is preferably amplified using normal DNA precursor nucleotides and at least one modified precursor nucleotide.

The term “amplifying” as used herein refers to any in vitro process for increasing the number of copies of a nucleotide sequence or sequences. Amplification of a target sample results in the incorporation of precursor nucleotides into the DNA being amplified.

Typically, amplification of a target sample is carried out using the polymerase chain reaction (PCR) using a combination of normal precursor nucleotides and modified precursor nucleotides. Normal precursor nucleotide(s) refer to the deoxyribonucleotides dATP, dCTP, dGTP and dTTP.

Modified precursor nucleotide(s) as used herein refers to a modified nucleotide or nucleotides that can be incorporated into a nucleic acid so that a substrate base or bases (endonuclease substrate base) is generated which is recognised by a DNA endonuclease enzyme.

According to one embodiment, a nucleotide containing the modified base partially replaces a normal precursor nucleotide.

According to an alternative embodiment, a nucleotide containing the modified base totally replaces a normal precursor nucleotide.

The amplification will typically involve amplifying a target nucleic acid using a combination of normal DNA precursor nucleotides and one or more modified precursor nucleotide(s) where a modified precursor nucleotide replaces all or a proportion of one of the normal precursor nucleotides.

Amplification of a nucleic acid using a modified precursor nucleotide to totally replace one of the normal precursor nucleotides results in the incorporation of an endonuclease substrate base into DNA in place of one of the four normal bases G, A, T, C. This permits cleavage of a primer extended in an amplification reaction at the first position 3′ of the primer where a normal base is replaced by modified base.

Following the amplification process, the amplified product is treated with a suitable DNA endonuclease enzyme which recognises the modified base and cleaves the DNA phosphodiester at or near the site of incorporation of said modified base. Prior to treatment with a suitable DNA endonuclease, double stranded DNA may be treated with exonuclease I. This treatment serves to digest any unused primers and any non-specific single stranded DNA amplification products, thus improving the signal to noise ratio. However, this is not an absolute requirement for the method according to the invention.

In the case where the modified precursor nucleotide is dITP, the modified base inosine will be generated at guanine positions in the amplified target nucleic acid sample. Addition of Endonuclease V to the sample cleaves the DNA predominantly at the second phosphodiester bond 3′ to the inosine, however the inosine residue is not removed from the DNA by the enzyme.

Although inosine has been termed and used as a universal base in that it can substitute for all four normal bases and base pairs with all four normal bases, it primarily acts as a substitute for guanine. Therefore in the method according to the invention, inosine primarily substitutes for guanine.

In the case where the modified precursor nucleotide is dUTP, the modified base uracil will be generated at thymine positions in the amplified target nucleic acid sample. Addition of Endonuclease V to the sample also cleaves the DNA at the second phosphodiester bond 3′ to the uracil base, however the uracil residue is not removed from the DNA by the enzyme, as discussed further below.

According to a further alternative embodiment, the modified base is introduced by chemical modification of an existing base.

The modified base can be introduced by chemical modification of a nucleic acid. Several methods exist where the treatment of DNA with specific chemicals modifies existing bases so that they are recognised by specific DNA endonuclease enzymes. For example, treatment of DNA with sodium bisulfite causes deamination of cytosine residues in DNA to form uracil residues in DNA, which target Endonuclease V cleavage of the DNA strands containing uracil residues. Treatment of DNA with oxidising agents, generates several modified bases including inosine, caused by deamination of adenine. Thus, bases present in the nucleic acid can be converted into modified bases by chemical means.

The modified base is preferably selected from inosine (hypoxanthine) and uracil.

The preferred modified precursor nucleotides dITP and dUTP when incorporated into DNA generate the endonuclease substrate bases, inosine and uracil, respectively.

The modified precursor nucleotide dUTP is a base sugar phosphate comprising the base uracil and a sugar phosphate moiety.

The modified precursor nucleotide dITP is a base sugar phosphate comprising the base hypoxanthine and a sugar phosphate moiety.

Amplification of a target nucleic acid using the precursor nucleotides dATP, dCTP, dGTP, dTTP and a specified amount of the modified precursor nucleotide dITP results in an amplified DNA where guanine is preferentially replaced randomly by hypoxanthine. The hypoxanthine is incorporated in the newly synthesised DNA strand at positions complementary to cytosine residues in the template DNA strand during the amplification process when the other precursor nucleotides are not limiting.

Amplification of a target nucleic acid using the precursor nucleotides dATP, dCTP, dGTP, dTTP and a specified amount of the modified precursor nucleotide dUTP results in an amplified DNA where thymine is replaced randomly by uracil. The uracil is incorporated in the newly synthesised DNA strand at positions complementary to adenine residues in the template DNA strand during the amplification process.

More than one type of modified nucleotide can be incorporated. For example, dUTP can replace dTTP and dITP can replace dGTP.

In one embodiment of the invention, the endonuclease is a thermostable endonuclease.

Thermostable endonucleases are available from several thermostable organisms including Endonuclease V-like activity from Thermatoga maritima and Archeaglobus fulgidus.

A DNA endonuclease as used herein cleaves the DNA backbone at or near the site of the endonuclease substrate base but predominantly at the second phosphodiester bond 3′ of the modified base. When the endonuclease cleaves the DNA backbone at or near all the sites of incorporation of the endonuclease substrate base many different fragments are generated.

The method of cleavage is referred to herein as endonuclease-mediated cleavage.

A specific example of an endonuclease as used herein is Endonuclease V from E. coli (also called Deoxyinosine 3′-Endonuclease) which cleaves at the second phosphodiester bond on the 3′ side of the endonuclease substrate base. This is distinct from some other endonucleases that cut at the site of the base and also distinct from glycosylases which excise glycosylase substrate bases. It should be noted that some endonuclease substrate bases can also be glycosylase substrate bases. For example, uracil and inosine are endonuclease substrate bases which target cleavage of DNA by Enonuclease V. However, these bases are also substrates for Uracil DNA glycosylase and 3-methyl-adenine glycosylase, respectively. Endonuclease V cleaves 3′ or downstream of the modified base, therefore an upstream fragment is generated that contains the modified base.

Fragments of differing lengths are therefore produced if there are differences in the sequence of the DNA due to additional or reduced incorporation of an endonuclease substrate base.

The method according to the invention is distinguishable from the methods described and claimed in WO 97/03210 and WO 99/54501 in that in the case of the cleavage step of the present invention a single enzyme recognises and cleaves the DNA in a single step, in contrast with the requirement for two separate enzymes or an enzyme and a chemical agent in the case of the prior art methods. In addition, in the case of the present invention, the modified base remains in the upstream fragment, whereas, in the case of the prior art, the modified base is excised from the DNA.

In one embodiment of the invention, the endonuclease cleavage reaction is carried out concurrently with a thermocycled amplification reaction using a thermostable endonuclease.

In one embodiment of the invention, one of the initial primers, termed the diagnostic primer, is placed adjacent to a locus where a DNA sequence variation such as a polymorphism or a mutation occurs, so that the first modified base incorporated into the extended primer is at the mutation locus, then the initial primer will be extended during the amplification reaction and then cleaved to a specific length depending on whether or not a mutation is present at the mutation locus following amplification and endonuclease-mediated cleavage. The fragments generated following the initial extension and endonuclease-mediated cleavage, containing the modified base are referred to as upstream fragments. The primers prior to extension are referred to as the initial primers in this embodiment, one of which is termed the diagnostic primer.

Thus, in an important aspect of the invention, at least one of the primers for the amplification step, i.e. the diagnostic primer, is positioned adjacent a locus where a DNA sequence variation occurs.

In a further embodiment of the invention, the diagnostic primer is placed adjacent to a locus where a DNA sequence variation such as a polymorphism or a mutation occurs (upstream), so that the first modified base incorporated into the extended primer is after the polymorphic locus (downstream), then the initial primer will be extended and then cleaved to a similar length but will have a different molecular mass or hydrophobicity depending on whether or not a polymorphism is present at the polymorphic locus following amplification and endonuclease-mediated cleavage.

This mass or hydrophobicity difference is readily determined by analysis on a mass spectrometer or HPLC, respectively. Alternatively, the sequence difference can be determined by differential hybridization of the upstream fragments.

In a still further embodiment of the invention, the method herein defined can be used to scan a stretch of nucleic acid for sequence variations. In this embodiment the initial primers used to amplify the stretch of nucleic acid, for example in PCR, are placed surrounding the area to be screened for genetic variations such as a polymorphism or a mutation so that the modified base is incorporated into the extended primer at all positions of the normal nucleotide that it is replacing. Endonuclease mediated cleavage of the amplified DNA will yield a population of fragments of various lengths dependent on the position of the replaced normal nucleotide in the original nucleic acid and also dependent on the sequence of the genetic variation. Therefore, the presence or absence of a sequence variation is determined by a gain or loss of one or more of the fragments or, indeed, a change in mass or hydrophobicity of one or more fragments.

When a proportion of a normal precursor nucleotide is replaced with a modified precursor nucleotide in the amplification reaction, endonuclease mediated cleavage of the amplified DNA will yield a population of upstream fragments of various lengths, since different molecules will be cleaved at different points depending on where the modified precursor nucleotide was incorporated. The length of each fragment is determined by the position of incorporation of the modified precursor nucleotide during extension from the 3′ end of the primer.

When a modified precursor nucleotide replaces a proportion of one of the normal precursor nucleotides, preferably the ratio of the modified precursor nucleotide to the normal precursor nucleotide that it is replacing is such that an optimum of one modified precursor nucleotide is incorporated per strand of amplified DNA. This allows subsequent cleavage of the amplified DNA strand into two fragments following action of an endonuclease that is specific for that modified base. A higher ratio of modified base to normal base is used to generate more than one cleavage site pelt amplified DNA strand.

The presence of a sequence variation or mutation results in the appearance or disappearance of a cleavage fragment as judged by comparison with the known DNA sequence of the amplified molecule. Size and/or mass analysis of the fragments allows the precise location and sequence of the mutation in the target nucleic acid sample to be determined. Therefore, if a sequence variation occurs such that an additional site of modified precursor nucleotide incorporation is generated, an additional cleavage fragment will be observed upon analysis of the ladder of cleavage products. If a sequence variation occurs such that a site of modified precursor nucleotide incorporation is lost, the corresponding cleavage fragment will not be observed upon analysis of the ladder of cleavage products.

If the sequence variation occurs such that it does not cause the gain or loss of a modified nucleotide incorporation site, the variation can be detected and determined by the change in mass of the upstream fragment that contains said variation. Alternatively, said variation can be detected by observing the altered electrophoretic mobility of said upstream fragment under non-denaturing conditions.

Direct detection of upstream fragments can be achieved by a variety of means. The initial primer used in the extension may be suitably labelled.

Labelling of the primers can be performed by a variety of means including addition of a radioactive, fluorescent, or detectable ligand to the primer during or post primer synthesis. The use of a labelled precursor nucleotide or dideoxy terminator nucleotide in any of the extension reaction facilitates detection of the upstream fragments.

Direct DNA staining methods such as silver or ethidium bromide staining facilitate detection of all products after size separation, for example after separation by electrophoresis.

All DNA fragments can be detected, including upstream fragments, by incorporation of a detectable ligand during the initial amplification reaction, and/or during a subsequent extension reaction, if such is carried out. Such labelling can be achieved by using labelled nucleotides during the polymerisation steps, for example, using fluorescent labelled nucleotides in a manner known per se.

The products/cleavage pattern resulting from the endonuclease mediated cleavage of the amplified target nucleic acid sample may be analysed by existing DNA sizing methods, such as polyacrylamide gel electrophoresis or high performance liquid chromatography (HPLC).

When the method according to the invention is used for genotyping a known polymorphism, the associated upstream fragment determines the presence of one or other of the alleles.

When the method according to the invention is used for scanning for polymorphism, comparison of the analysis with the wild type DNA allows determination of the presence or absence of a sequence variation in the target nucleic acid sample.

Labelling of the upper primer prior to the amplification process facilitates detection of the upstream fragments derived from the upper primer. On the other hand, labelling of the lower primer prior to the amplification process facilitates detection of the upstream fragments derived from the lower primer. The use of labelled precursor nucleotides in the initial amplification process and/or during a subsequent extension reaction, when such is carried out, facilitates detection of all the fragments generated as a result of the endonuclease mediated cleavage of the amplified target nucleic acid sample.

An alternative method for detecting the cleaved DNA fragments, including upstream and downstream fragments, is by hybridisation, using a probe which is a labelled nucleic acid fragment. For example, the upstream fragments of the amplified upper strand can be detected by hybridisation of a suitably labelled oligonucleotide that is complementary in sequence to the upper primer. Similarly, the upstream fragments of the amplified lower strand can be detected by hybridisation of a suitably labelled oligonucleotide that is complementary in sequence to the lower primer.

It will be appreciated that using certain conventional detection methods, no additional label is required in order to detect the DNA. Such a detection method would be mass spectrometry. Using this method, all cleavage fragments are detected directly and separated based on their mass. This method has the added advantage in that, for example, upstream fragments of the same length can be identified and distinguished on the basis that they have a different mass due to their different base composition.

Another such detection method is UV absorption.

The 3′ terminal sequence of the upstream fragment is enzymatically synthesised herein and is directly related to the nucleic acid being characterised since the target nucleic acid has acted as the template for its synthesis. Thus the 3′ end of the upstream fragment is complementary to the target nucleic acid. Accordingly, determination of the nature of the 3′ end of the upstream fragment by any means allows characterisation of the nucleic acid from which it was derived.

Such upstream fragments are generated so that they can be used as primers for a subsequent extension reaction, if required.

Accordingly, the upstream fragment generated in step ii) can be used as a primer for a subsequent extension reaction.

The extension can be carried out using a DNA polymerase.

Alternatively, the extension can be carried out using a DNA ligase.

Thus, the nature of the sequence of the 3′ end of upstream fragments can be determined by their ability to function as primers in a subsequent extension reaction using a template nucleic acid. Essentially, such determination is based on the ability of the 3′ end of the upstream fragment to hybridise to a selected template under selected conditions. Following partial or complete hybridisation the upstream fragment may be extended using a nucleic acid polymerase and nucleic acid precursors or selected combinations of same. It will be appreciated that multiple possibilities exist for the selection of template molecules. Nonetheless, the extension of the upstream fragment is a measure of its ability or lack of ability to hybridise to a selected template molecule and thus the determination of the nature of the sequence of the 3′ end of upstream fragment is on this basis.

Because the 3′ terminus of the upstream fragments differs depending on whether a mutation was present or absent at the mutation locus, be it the ultimate 3′ terminal nucleotide or, indeed, the last one to five or so 3′ terminal nucleotides, analysis of the ability of the upstream fragment to function in a subsequent extension reaction using a template nucleic acid permits the determination of whether a mutation was present or absent at the mutation locus. Any naturally occurring or enzymatically or chemically synthesised template which fully or partially hybridises to the upstream fragment can be designed and/or selected as a template nucleic acid allowing the ability of the upstream fragment to function as a primer to be determined.

As indicated above, the nature of the sequence of the 3′ end of upstream fragments can also be determined using extension by DNA ligase. In this aspect of the invention, the upstream fragment and a capture or reporter oligonucleotide are hybridised adjacent to one another on a template DNA strand. The ligase forms a phosphodiester bond between the upstream fragment and the reporter oligonucleotide, provided that the upstream fragment and the oligonucleotide junction have correctly hybridised with the template, i.e. no mismatches or gaps present at the junction. Thus, an exact match between the oligonucleotides and the template DNA at the junction permits the ligation of the upstream fragment to the reporter oligonucleotide forming a larger product which is the cumulative size of both the oligonucleotides. This reaction could also be cycled in a ligase chain reaction resulting in the amplification of the larger ligated product. Thus, detection of the larger product shows that the 3′ end of the upstream fragment hybridised to the template strand at the junction, while the absence of a larger product indicates that there were differences between the upstream fragment sequence and the sequence of the template DNA.

As indicated above, the endonuclease cleavage reaction can also be carried out simultaneously with a thermocycled polymerization reaction which incorporates the modified base into the DNA. This can be achieved by including a thermostable Endonuclease V-like enzyme in the thermocycled amplification reaction. For example, one can carry out the initial amplification of target nucleic acid using only normal precursor nucleotides dGTP, dATP, dTTP and dCTP. The product of this reaction is then treated with a phosphatase to degrade all nucleotide triphosphates and the phosphatase is then heat denatured. Fresh nucleotides are then added, where at least one normal nucleotide is partially or completely replaced by a modified precursor nucleotide, in addition to thermostable polymerase, thermostable endonuclease and additional diagnostic primer and a thermocycled amplification reaction is effected. In effect, during each cycle, the diagnostic primer is extended using the initial amplification product as template and modified nucleotide is incorporated into the extended primer, thus generating a substrate for cleavage by the thermostable endonuclease, thereby generating upstream/diagnostic fragments. The diagnostic primer can be used as one of the original amplification primers or can be an alternative or nested primer. If the diagnostic primer is different from the initial amplification primers then an Exonuclease I step can also be included with the phosphatase treatment, thereby degrading all unused initial primers so that the diagnostic primer is the only primer present during the cycling reaction.

It will be appreciated that the present invention has significant advantages over the prior art, in particular, the prior art hereinabove referred to.

For example, the method according to the invention can be viewed as a single step reaction requiring just a single enzyme in the sense that no secondary cleavage enzyme or step is required, which will allow higher throughput and more cost-effective DNA characterisation. Upstream DNA fragments are directly extendible and no extra processing is needed.

One can use a single enzyme to detect all possible single base polymorphisms, for example, Endonuclease V from specific sources can cut at U and I sites, therefore both T to A (and A to T) and G to C (and C to G) polymorphisms can be detected.

When using an Endonuclease V-type of enzyme, there is no absolute need for Exonuclease I treatment of the amplified product prior to endonuclease mediated cleavage since any diagnostic fragment will be larger than the unused primer, even if primer must be designed adjacent to the polymorphic site, since Endonuclease V cuts two phosphodiester bonds downstream of the polymorphic site.

The method according to the invention allows one to carry out both genotyping and the discovery of polymorphisms.

As indicated above, the method according to the invention permits detection of polymorphisms in any sequence context and said detection is not dependent of surrounding nucleotide sequence.

The method according to the invention is not dependent on the presence of mismatches or external probes for detection of polymorphisms.

### BRIEF DESCRIPTION OF THE DRAWING

The accompanying FIGURE is a diagrammatic representation of the method in accordance with the invention.

### MODES FOR CARRYING OUT THE INVENTION

### EXAMPLE 1

The method according to the invention was used to detect the presence of a G to C SNP (single nucleotide polymorphism) at position 1357 of the human pyruvate dehydrogenase kinase, isoenzyme 2 gene (PDK2) (Accession number L42451) in a patient heterozygous for that SNP. The sequence of steps is depicted in the accompanying FIGURE. The lower strand shown in the FIGURE is selected as the target strand and the presence or absence of a G nucleotide at the SNP site was determined. The G allele refers to that PDK2 allele bearing the G nucleotide at position 1357 (on the upper strand) and the C allele refers to the PDK2 allele bearing the C nucleotide at position 1357 (on the upper strand).

The diagnostic primer (in this case it is the lower primer) was synthesised with a 5′ fluorescent label (denoted by *). The genomic DNA sample was amplified by PCR as follows: the reaction mix for PCR contained 10 ng genomic DNA from the patient, 0.2 mM dATP, dCTP, dITP and dTTP, 1.5 mM MgCl₂, 10 mM Tris-HCl, pH8.3, 500 mM KCl, 0.3 units JumpStart Taq Polymerase, 6 pmoles of each primer in a total volume of 5 μl. The reaction was then placed in a thermocylcer with a heated hood. 35 Cycles of 95° C. for 30 seconds, 45° C. for 60 seconds and 65° C. for 300 seconds were carried out in a thermocycler followed by 65° C. for 300 seconds. The reaction mixture bearing the amplified DNA was then treated with Exonuclease I to digest any unincorporated primers remaining in the reaction mixture as follows: 1 μl of the PCR reaction with 2 units of Exonuclease I in a 5 μl reaction volume at 37° C. for 30 minutes. The exonuclease was subsequently heat inactivated by incubating the reaction at 80° C. for 15 minutes.

The reaction volume was increased to 20 μl with the addition of H₂O and was purified by centrifugation through a CentriSep (CentriSep is a Trade Mark) DNA spin column (Princeton Separations). The purified DNA (20 μl) was then digested with 1 μl of Endonuclease V (0.4 μg/ml preparation) in the presence of 2 mM MgCl₂. The reaction was incubated at 65° C. for 30 minutes. 2 μl of the reaction was added to 101 of formamide and 0.2 μl of ROX labeled size standard, which was heated to 95° C. for 5 minutes.

The reaction was loaded onto an ABI 3100 Genetic Analyzer containing POP-4 (POP is a Trade Mark) matrix and electrophoresed at 15 kV for 30 minutes. Analysis of the run showed that two cleavage products were present in approximately equal amounts. One product was 29 nucleotides (n) in length, corresponding to the G allele, while the second product corresponding to the C allele, was 23 n in size, as described below.

The 23 n product could only have been generated if an inosine residue was incorporated in the lower strand at position 1357. Thus, the detection of a 23 n fragment is diagnostic of the presence of a C residue at position 1357 on the upper strand, hence incorporation of an inosine residue as the complementary base on the lower strand and the Endonuclease V enzyme cutting the lower strand one nucleotide 3′ (i.e. downstream) of the inosine residue. In the G allele, the first inosine incorporated on the lower strand should be opposite the C nucleotide at position 1350 on the upper strand and should result in a 29 n product since Endonuclease V enzyme cuts the lower strand one nucleotide 3′ (i.e. downstream) of the inosine residue. Thus the detection of a 29 n fragment is diagnostic of the absence of a C residue at position 1357 on the upper strand, thereby indicating the presence of the other allele, namely G allele at this position. The 23 n and 29 n products were detected in roughly equal amounts indicating that the target nucleic acid was from an individual heterozygous for the G1357C polymorphism. When a patient homozygous for the G allele was analysed, then only a 29 n product was detected. When a patient homozygous for the C allele was analysed, then only the 23 n product was detected. The relative intensity of the 29 n product to the 23 n product allows one to determine the relative levels of the normal and mutant allele in a target nucleic acid sample. This is especially useful for analyzing pooled DNA samples where there may be a large difference between the levels of each allele.

To diagnose a G to C transversion, or vice versa, the target strand can be either strand of the target locus. The decision on which strand the diagnostic primer is designed against can be made based upon optimal primer design considerations and on the most desirable product lengths.

### EXAMPLE 2

The method according to the invention was used to detect the presence of a G to A SNP at position 380 of the Synaptopysin gene from Atlantic cod (Gadus morhua) in an individual heterozygous for that SNP. The sequence of steps is similar to that depicted in the accompanying FIGURE. However, in this instance the upper strand is selected as the target strand and the presence or absence of a G nucleotide at the SNP site was determined. The G allele refers to that synaptophysin allele bearing the G nucleotide at position 380 and the A allele refers to the synaptophysin allele bearing the A nucleotide at position 380.

The diagnostic primer (in this case it is the upper primer) was labelled with a γ³²PATP. The genomic DNA sample was amplified by PCR as follows: the reaction mix for PCR contained 100 ng genomic DNA from the Atlantic cod, 0.2 mM dATP, dCTP, dITP and dTTP, 1.5 mM MgCl₂, Bioline (Bioline is a Trade Mark) Taq buffer, and 1 unit of Bioline Taq Polymerase, 50 ng of each primer in a total volume of 25 μl. The reaction was then placed in a thermocylcer with a heated hood. 35 Cycles of 95° C. for 60 seconds, 45° C. for 60 seconds and 65° C. for 180 seconds were carried out in a thermocycler followed by 65° C. for 600 seconds. The reaction mixture bearing the amplified DNA was then treated with Exonuclease I to digest any unincorporated primers remaining in the reaction mixture as follows: 5 μl of the PCR reaction with 2 units of Exonuclease I, 5 mM MgCl₂, 10 ml Tris/HCl pH 7.5 in a 19 μl reaction volume at 37° C. for 30 minutes. The exonuclease was subsequently heat inactivated by incubating the reaction at 80° C. for 15 minutes.

The DNA mix (19 μl) was then digested with 1 μl of Endonuclease V (0.4 μg/ml preparation). The reaction was incubated at 65° C. for 30 minutes. 20 μl of formamide dye was added to the reaction, which was heated to 95° C. for 5 minutes.

The reaction was loaded onto a 6% denaturing gel and electrophoresed at 60 W for 90 minutes. Following electrophoresis the gel was dried and exposed to a phosphor screen for 120 minutes and analysed using Storm 860 (Storm is a Trade Mark) (Molecular Dynamics). Analysis of the run showed that two cleavage products were present in approximately equal amounts. One product was 23 nucleotides (n) in length, corresponding to the G allele, while the second product corresponding to the A allele, was 26 n in size, as described below. Since the 23 n and 26 n product were detected in roughly equal amounts, this indicates that the target nucleic acid was from an individual fish heterozygous for the G380A polymorphism.

The 23 n product could only have been generated if an inosine residue was incorporated in the upper strand at position 380. Thus the detection of a 23 n fragment is diagnostic of the presence of a G residue at position 380 on the upper strand. In the A allele, the first inosine incorporated on the upper strand should be the nucleotide at position 383 on the upper strand and should result in a 26 n product since Endonuclease V enzyme cuts the one nucleotide 3′ (i.e. downstream of the inosine residue. Thus, the detection of a 26 n fragment is diagnostic of the absence of a G residue at position 380 on the upper strand, thereby indicating the presence of the other allele, namely A allele at this position. When a fish which was homozygous for the G allele was analysed, then only a 23 n product was detected. When a fish homozygous for the A allele was analysed, then only the 26 n product was detected.

### EXAMPLE 3

The method according to the invention was used to visualise the presence of the G (and therefore C also) content of a segment of the synaptopysin gene from Atlantic cod (Gadus morhua). This example describes where the upper strand is selected as the target strand and the position of G nucleotides was determined.

The upper primer was labelled with a γ³²PATP. The genomic DNA sample was amplified by PCR as follows: the reaction mix for PCR contained 10 ng genomic DNA from the cod, 0.2 mM dATP, dCTP, and dTTP, 0.05 mM dGTP and 0.15 mM dITP, 1.5 mM MgCl₂, Bioline Taq buffer, and 1 unit of Bioline Taq Polymerase, 6 pmol of each primer in a total volume of 25 μl. The reaction was then placed in a thermocylcer with a heated hood. 35 Cycles of 95° C. for 60 seconds, 45° C. for 60 seconds and 65° C. for 60 seconds were carried out in a thermocycler followed by 65° C. for 600 seconds. The reaction mixture bearing the amplified DNA was then treated with Exonuclease I to digest any unincorporated primers remaining in the reaction mixture as follows: 5 μl of the PCR reaction with 2 units of Exonuclease I, 5 mM MgCl₂, 10 mM Tris/HCl pH 7.5 in a 19 μl reaction volume at 37° C. for 30 minutes. The Exonuclease I was subsequently heat inactivated by incubating the reaction at 80° C. for 15 minutes.

The DNA mix (19 μl) was then digested with 1 μl of Endonuclease V (0.4 μg/ml preparation). The reaction was incubated at 65° C. for 30 minutes. 20 μl of formamide dye was added to the reaction, which was heated to 95° C. for 5 minutes.

The reaction was loaded onto a 6% denaturing gel and electrophoresed at 60 W for 90 minutes. Following electrophoresis the gel was dried and exposed to phosphor screen for 120 minutes and analysed using Storm 860 (Molecular Dynamics). Analysis of the run showed that cleavage products, i.e. bands visible on a gel, were present for each G residue in the sequence.

The ratio of I:G ensures that in every amplification, a proportion of the fragments will have an I residue at at least one G position in the fragment, so that the resulting test sample will provide a heterogenous mix of DNA fragments with I residues at all possible G positions. The resulting amplified strands are cleaved by Endo V and the resulting fragments are analysed by denaturing PAGE. Analysis of the resulting banding pattern allows for identification and localisation of a sequence change. For example, analysis of a normal DNA fragment containing 10 G residues will yield a PAGE ladder of 10 labelled fragments. Analysis of a similar fragment with a G-A mutation will result in the generation of a PAGE ladder of 9 fragments with the absence of a labelled fragment corresponding to that G residue that was mutated. Inosine in this instance is not incorporated at the mutation site and thus cleavage does not occur. Therefore a DNA fragment of this particular size is not generated. Alternatively if the sequence contains a mutation which results in an extra G residue, an extra labelled fragment will be observed in the PAGE ladder. The length of this additional fragment allows localisation of the sequence change.

### EXAMPLE 4

The method according to the invention was used to detect the presence of a G to T SNP (rs1894702) at position 101663 of the human Factor V (Accession number NT_{—}004668) in a patient heterozygous for that SNP. The sequence of steps is as generally depicted in the accompanying FIGURE. The upper strand in this instance was selected as the target strand and the presence or absence of a G nucleotide at the SNP site was determined. The G allele refers to that Factor V allele bearing the G nucleotide at position 101663 (on the upper strand), and the T allele refers to the Factor V allele bearing the T nucleotide at position 101663 (on the upper strand).

The upper primer was synthesised with a 5′ fluorescent label (FAM). The genomic DNA sample was amplified by PCR as follows: the reaction mix for PCR contained 10 ng genomic DNA from the patient, 0.2 mM dATP, dCTP, dGTP and dTTP, 1.5 mM MgCl₂, 10 mM Tris-HCl, pH8.3, 50 mM KCL, 0.3 units JumpStart Taq Polymerase, 6 pmoles of each primer in a total volume of 5 μl. The reaction was then placed in a thermocylcer with a heated hood. 35 Cycles of 95° C. for 30 seconds, 60° C. for 15 seconds and 72° C. for 15 seconds were carried out in a thermocycler followed by 72° C. for 300 seconds. The reaction mixture bearing the amplified DNA was then treated with Shrimp Alkaline Phosphatase (SAP) to digest any unincorporated nucleotides remaining in the reaction mixture as follows: 1 μl of the PCR reaction, and 1 unit SAP in a 5 μl reaction volume at 37° C. for 30 minutes. The SAP was subsequently heat inactivated by incubating the reaction at 80° C. for 15 minutes. The Exo I treated PCR was then diluted 500 fold, and 1 μl re-amplified as follows: 0.2 mM dATP, dCTP, dITP and dTTP, 1.5 mM MgCl₂, 10 mM Tris-HCl, pH8.3, 50 mM KCl, 0.3 units JumpStart Taq Polymerase, 6 pmoles of each primer in a total volume of 10 μl. The reaction was then placed in a thermocylcer with a heated hood, incubated at 95° C. for 180 seconds, followed by 45 cycles of 94° C. for 120 seconds, 45° C. for 60 seconds, 65° C. for 120 seconds, were carried out in a thermocycler followed by 65° C. for 600 seconds.

The reaction mixture bearing the amplified DNA was then treated with Exonuclease I to digest any unincorporated primers remaining in the reaction mixture as follows: 1.5 μl of the PCR reaction, and 2 units of Exonuclease I in a 5 μl reaction volume at 37° C. for 30 minutes.

The reaction volume was increased to 20 μl with the addition of H₂O and was purified by centrifugation through a CentriSep (CentriSep is a Trade Mark) DNA spin column (Princeton Separations). The purified DNA (20 μl) was then digested with 1.5 μl of Endonuclease V in the presence of 2 mM MgCl₂. The experiment was repeated with Endonuclease V from Archaeoglobus fulgidus and Thermatoga maritima with similar results) (0.4 μg/ml preparation). The reaction was incubated at 65° C. for 30 minutes for Thermatoga maritima and 85° C. for 30 minutes for Archaeoglobus fulgidus. 1.5 μl of the reaction was added to 10 μl of formamide and 0.21 μl of ROX labeled size standard, which was heated to 95° C. for 5 minutes.

The reaction was loaded onto an ABI 3100 Genetic Analyzer containing POP-4 (POP is a Trade Mark) matrix and electrophoresed at 15 kV for 30 minutes. Analysis of the run showed that two cleavage products were present in approximately equal amounts. One product was 21 nucleotides (n) in length, corresponding to the G allele, while the second product corresponding to the T allele, was 23 n in size, as described below.

The 21 n product could only have been generated if an inosine residue was incorporated in the upper strand at position 101663. Thus the detection of a 21 n fragment is diagnostic of the presence of a G residue at position 101663 on the upper strand by incorporation of an inosine at this site and the Endonuclease V enzyme cutting the upper strand one nucleotide 3′, i.e. downstream, of the inosine residue. In the T allele, the first inosine incorporated on the upper strand should be at position 101665 on the upper strand and should result in a 23 n product since Endonuclease V enzyme cuts the upper strand one nucleotide 3′ of the inosine residue. Thus the detection of a 23 n fragment is diagnostic of the absence of a G residue at position 101663 on the upper strand, thereby indicating the presence of the other allele, namely T allele at this position. The 21 n and 23 n product were detected in equal amounts indicating that the target nucleic acid was from an individual heterozygous for the rs1894702 polymorphism. When a patient homozygous for the G allele was analysed, then only a 21 n product was detected. When a patient homozygous for the T allele was analysed, then only the 23 n product was detected. The relative intensity of the 21 n product to the 23 n product allows one to determine the relative levels of each allele in a target nucleic acid sample. This is especially useful for analyzing pooled DNA samples where there may be a large difference between the levels of each allele.

### EXAMPLE 5

The method according to the invention was used to detect the presence of a G to C SNP (rs1042714) at position 4251 of the human Beta-2 adrenergic receptor (BAR2) (Accession number NT_{—}031801) in a patient heterozygous for that SNP. The sequence of steps is as generally depicted in the accompanying FIGURE. The upper strand in this instance is selected as the target strand and the presence or absence of a G nucleotide at the SNP site was determined. The G allele refers to that BAR2 allele bearing the G nucleotide at position 4251 (on the upper strand), and the C allele refers to the BAR2 allele bearing the C nucleotide at position 4251 (on the upper strand).

The upper primer (20 nucleotides) was synthesised with a 5′ fluorescent label (FAM). The genomic DNA sample was amplified by PCR as follows: the reaction mix for PCR contained 10 ng genomic DNA from the patient, 0.2 mM dATP, dCTP, dGTP and dTTP, 1.5 mM MgCl₂, 10 mM Tris-HCl, pH8.3, 50 mM KCl, 0.3 units JumpStart Taq Polymerase, 6 pmoles of each primer in a total volume of 5 μl. The reaction was then placed in a thermocylcer with a heated hood. 35 Cycles of 95° C. for 30 seconds, 60° C. for 15 seconds and 72° C. for 15 seconds were carried out in a thermocycler followed by 72° C. for 300 seconds. The reaction mixture bearing the amplified DNA was then treated with Shrimp Alkaline Phosphatase (SAP) to digest any unincorporated nucleotides remaining in the reaction mixture as follows: 1 μl of the PCR reaction, and 1 unit SAP in a 5 μl reaction volume at 37° C. for 30 minutes. The SAP was subsequently heat inactivated by incubating the reaction at 80° C. for 15 minutes. The PCR reactions were then diluted 500 fold, and re-amplified as follows: 0.2 mM dATP, dCTP, dITP and dTTP, 1.5 mM MgCl₂, 10 mM Tris-HCl, pH8.3, 50 mM KCl, 0.3 units JumpStart Taq Polymerase, 6 pmoles of each primer in a total volume of 10 μl. The reaction was then placed in a thermocylcer with a heated hood. 45 Cycles of 95° C. for 180 seconds, 94° C. for 120 seconds, 45° C. for 60 seconds, 65° C. for 120 seconds, were carried out in a thermocycler followed by 65° C. for 600 seconds.

The reaction mixture bearing the amplified DNA was then treated with Exonuclease I to digest any unincorporated primers remaining in the reaction mixture as follows: 1.5 μl of the PCR reaction, and 2 units of Exonuclease I in a 5 μl reaction volume at 37° C. for 30 minutes.

The reaction volume was increased to 20 μl with the addition of H₂O and was purified by centrifugation through a CentriSep DNA spin column (Princeton Separations). The purified DNA (20 μl) was then digested with 1.5 μl of Endonuclease V in the presence of 2 mM MgCl₂. The experiment was repeated with Endonuclease V from Archaeoglobus fulgidus and Thermatoga maritima with similar results) (0.4 μg/ml preparation). The reaction was incubated at 65° C. for 30 minutes for Thermatoga maritima and 85° C. for 30 minutes for Archaeoglobus fulgidus. 1.5 μl of the reaction was added to 101 μl of formamide and 0.2 μl of ROX labeled size standard, which was heated to 95° C. for 5 minutes.

The reaction was loaded onto an ABI 3100 Genetic Analyzer containing POP-4 matrix and electrophoresed at 15 kV for 30 minutes. Analysis of the run showed that two cleavage products were present in approximately equal amounts. One product was 22 nucleotides (n) in length, corresponding to the G allele, while the second product corresponding to the C allele, was 26 n in size, as described below.

The 22 n product could only have been generated if an inosine residue was incorporated in the upper strand at position 4251. Thus the detection of a 22 n fragment is diagnostic of the presence of a G residue at position 4251 on the upper strand, by incorporation of an inosine residue and the Endonuclease V enzyme cutting the upper strand one nucleotide 3′ i.e. downstream of the inosine residue. In the C allele, the first inosine incorporated on the upper strand is at position 4255 on the upper strand and should result in a 26 n product since Endonuclease V enzyme cuts the upper strand one nucleotide 3′ of the inosine residue. Thus the detection of a 26 n fragment is diagnostic of the absence of a G residue at position 4251 on the upper strand, thereby indicating the presence of the other allele, namely C allele at this position. The 22 n and 26 n product were detected in equal amounts indicating that the target nucleic acid was from an individual heterozygous for the rs1042714 polymorphism.

1. A method for characterising nucleic acid molecules, which comprises the steps of:
i) introducing a modified base which is a substrate for a DNA endonuclease into a target DNA molecule; and
ii) reacting the nucleic acid containing the modified base with said DNA endonuclease such that the nucleic acid is cleaved to generate an upstream fragment containing said modified base and bearing a 3′ hydroxyl group.

2. A method according to claim 1, wherein the modified base is introduced by enzymatic amplification of the nucleic acid.

3. A method according to claim 1 or 2, wherein a nucleotide containing the modified base partially replaces a normal precursor nucleotide.

4. A method according to claim 1 or 2, wherein a nucleotide containing the modified base totally replaces a normal precursor nucleotide.

5. A method according to claim 1, wherein the modified base is introduced by chemical modification of an existing base.

6. A method according to any preceding claim, wherein the modified base is selected from inosine and uracil.

7. A method according to any preceding claim, wherein the endonuclease is a thermostable endonuclease.

8. A method according to any preceding claim, wherein the endonuclease cleavage reaction is carried out concurrently with a thermocycled amplification reaction using a thermostable endonuclease.

9. A method according to any one of claims 2-8, wherein at least one of the primers for the amplification step is positioned adjacent a locus where a DNA sequence variation occurs.

10. A method according to any one of claims 1-6 and 9, wherein the endonuclease is Endonuclease V from E. coli.

11. A method according to any preceding claim, wherein the upstream fragment generated in step ii) is used as a primer for a subsequent extension reaction.

12. A method according to claim 11, wherein the extension is carried out using a DNA polymerase.

13. A method according to claim 11, wherein the extension is carried out using a DNA ligase.

14. A method according to claim 1, substantially as herein before described and exemplified

15. A method according to any preceding claim for use in detecting polymorphisms and mutations.

16. A method according to any one of claims 1-14, for use in nucleic acid identification and profiling.

17. A method according to any one of claims 1-14, for rapidly genotyping known polymorphisms and mutations.

18. A method according to any one of claims 1-14, for scanning a nucleic acid sequence for the presence of known or unknown mutations and polymorphisms.
